(19) United States
(12) Patent Application Publication
(10) Pub. No.: US 2012/0157826 A1
(43) Pub. Date: Jun. 21, 2012

(54) Method for the Remote Detection of Wound Interface Materials Inside the Body

(76) Inventors: Christopher Brian LOCKE, Bournemouth (GB); Timothy Mark ROBINSON, Basingstoke (GB)

(21) Appl. No.: 13/323,320

(22) Filed: Dec. 12, 2011

Related U.S. Application Data
(60) Provisional application No. 61/423,531, filed on Dec. 15, 2010.

(51) Int. Cl. A61B 5/05

(52) U.S. Cl. 600/424

(57) ABSTRACT

Methods and apparatuses of detecting wound interface materials in a wound are provided. In particular, a metal detector may be used to detect parts of a metallic negative pressure manifold used in combination with negative pressure wound therapy.

### CROSS-REFERENCE TO RELATED APPLICATION

This application claims priority to U.S. Provisional Patent Application Ser. No. 61/423,531 filed Dec. 15, 2010. This provisional application is expressly incorporated by reference.

### BACKGROUND

1. Field of the Invention

Embodiments of the present invention relate generally to electronic methods for remotely detecting wound interface materials inside the body of a patient. More particularly, embodiments of the present invention relate to methods for detecting wound interface material in a wound using a metal detector.

2. Background Information

Clinical studies and practice have shown that providing a reduced pressure in proximity to a tissue site augments and accelerates the growth of new tissue at the tissue site. The applications of this phenomenon are numerous, but application of reduced pressure has been particularly successful in treating wounds. This treatment (frequently referred to in the medical community as “negative pressure wound therapy,” “reduced pressure therapy,” or “vacuum therapy”) provides a number of benefits, including faster healing and increased formulation of granulation tissue. Typically, reduced pressure is applied to tissue through a wound insert (e.g., a porous pad or other manifold device). The wound insert typically contains cells or pores that are capable of distributing reduced pressure to the tissue and channeling fluids that are drawn from the tissue. The wound insert can be incorporated into a wound dressing having other components that facilitate treatment, such as, for example, a drape (e.g., adhesive surgical drape). Instillation of fluids (e.g., irrigation fluids and/or medicaments) may be used in conjunction with negative pressure wound therapy to promote healing and/or improve efficacy.

### SUMMARY

The present disclosure includes embodiments of methods of detecting the presence of a negative pressure manifold device in a patient. In certain embodiments, the method comprises positioning a metal detector proximate to a wound surface of a patient; and monitoring the metal detector to determine if the metal detector provides an indication of the presence of a metal.

In other embodiments, the method may further comprise the step of removing a wound insert from a wound proximate to the wound surface prior to positioning the metal detector proximate to the wound surface of the patient.

In some embodiments the wound insert comprises metallic particles. In other embodiments, the metal detector may be configured to detect and identify a type of metal comprised in the metallic particles. In specific embodiments, the type of metal comprised in the metallic particles is silver. In some embodiments, the metal detector may further comprise a screen configured to display a message to a user.

Other embodiments of a method of detecting wound interface material in a wound may comprise the steps of obtaining a metal detector at a first sensitivity setting; positioning the metal detector at a first distance adjacent to a wound of a patient; actuating the metal detector; and observing whether metal is detected in the wound.

The method may further comprise the step of removing a wound interface material from the wound if metal is detected in the wound. In certain embodiments, the metal detector is a hand-held metal detector.

In particular embodiments, the wound may further comprise remnants of wound interface material, and the method may further comprise adjusting the metal detector to a second sensitivity setting, where the second sensitivity setting is more sensitive than the first sensitivity setting; actuating the metal detector; observing whether the metal detector detects metal in the wound; and removing remnants of wound interface material from the wound.

In other particular embodiments, the wound further comprises remnants of wound interface material, the method further comprising: positioning the metal detector at a second distance adjacent to the wound, the second distance being smaller than the first distance; actuating the metal detector; observing whether metal is detected in the wound; and removing remnants of wound interface material from the wound.

In certain embodiments, the removing step may further comprise surgically removing remnants of wound interface material from the wound. Other embodiments comprise the additional step of wetting the wound insert.

In certain embodiments, the metal detector has a volume less than 40 in³. The metal detector may be battery-operated in some embodiments. In other embodiments, the metal detector may be hand-held.

In some embodiments, the wound insert further comprises silver, iron, cobalt, gadolinium, europium, yttrium, chromium, or nickel.

In certain embodiments, the wound further may comprise remnants of wound interface material, and the method may comprise the additional steps of adjusting the metal detector to a second sensitivity setting, where the second sensitivity setting is more sensitive than the first sensitivity setting; actuating the metal detector; observing whether the metal detector detects metal in the wound; and removing remnants of wound interface material from the wound.

In still other embodiments, the wound may further comprise remnants of wound interface material, and the method may further the steps of positioning the metal detector at a second distance adjacent to the wound, the second distance being less than the first distance; actuating the metal detector; observing whether the metal detector detects metal in the wound; and removing remnants of wound interface material from the wound.

A medical device is presented in certain embodiments. In specific embodiments, a medical device is presented that comprises a metal detector configured to detect metal in a wound interface material proximal to a wound of a patient.

The device may further comprise an indicator configured to provide an indication when metal is detected in a wound interface material proximal to a wound in a patient in some embodiments. In certain embodiments, the indicator may emit an audible signal, visual signal, or may provide tactile feedback. In still other embodiments, the device may comprise a screen configured to display a message to a user that wound interface material has been detected.

In still other embodiments, a kit is presented. In specific embodiments, the kit may comprise a wound insert comprising a metal; and a metal detector configured to detect the metal comprised in the wound insert. The metal detector may further comprise an indicator configured to provide an indication when metal is detected in the wound insert. In certain embodiments, the indicator may emit an audible signal, visual signal, or may provide tactile feedback. In still other embodiments, the device may comprise a screen configured to display a message to a user that wound interface material has been detected.

Any embodiment of any of the present systems and/or methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 illustrates a schematic diagram of a negative pressure wound therapy system.

FIGS. 2A-2C illustrate wound interface materials being removed from a wound.

FIG. 3 illustrates one embodiment of a method for remotely detecting wound interface materials in wound.

### DESCRIPTION OF ILLUSTRATIVE EMBODIMENTS

The term “coupled” is defined as connected, although not necessarily directly, and not necessarily mechanically; two items that are “coupled” may be integral with each other. The terms “a” and “an” are defined as one or more unless this disclosure explicitly requires otherwise. The terms “substantially,” “approximately,” and “about” are defined as largely but not necessarily wholly what is specified, as understood by a person of ordinary skill in the art.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a wound-treatment method that “comprises,” “has,” “includes” or “contains” one or more steps possesses those one or more steps, but is not limited to possessing only those one or more steps. Likewise, a wound dressing that “comprises,” “has,” “includes” or “contains” one or more elements possesses those one or more elements, but is not limited to possessing only those elements. For example, in a wound dressing that comprises one of the present wound inserts and a drape, the wound dressing includes the specified elements but is not limited to having only those elements. For example, such a wound dressing could also include a connection pad configured to be coupled to a negative pressure wound therapy (NPWT) apparatus (e.g., including a vacuum source and/or a fluid source). As used herein, the terms “wound”, “wound surface” and related terms are to be interpreted broadly to include, for example, both surgical and non-surgical wounds and wound surfaces.

Further, a device or structure that is configured in a certain way is configured in at least that way, but it can also be configured in other ways than those specifically described.

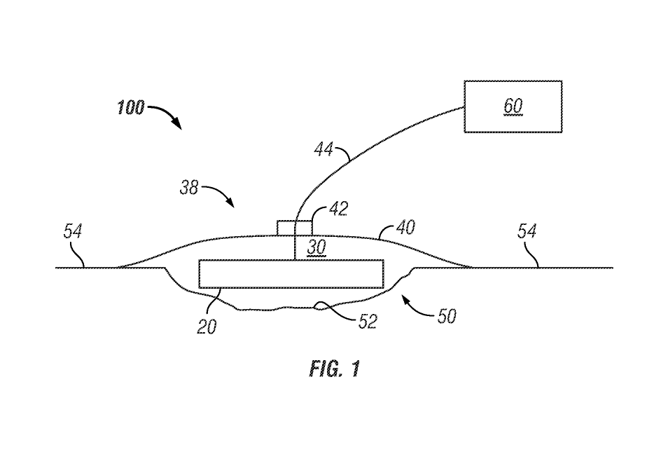

Referring now to the drawings, FIG. 1 illustrates a wound insert 20 as part of a wound treatment system 100. In the embodiment shown, wound insert 20 is shown in wound 50 of a patient (not shown) having a wound surface 52. A drape 40 is placed over wound 50 and wound insert 20 such that wound insert 20 is between drape 40 and wound 50, creating space 30. Drape 40 is coupled to the skin 54 of the patient. Wound insert 20 is coupled to a wound treatment apparatus 60 by conduit 44. Apparatus 60 may comprise a vacuum source configured to apply negative pressure to wound insert 20 through conduit 44. Apparatus 60 may further comprise a fluid source configured to deliver a fluid through conduit 44 to wound insert 20. Examples of such fluids include a medicinal fluids, antibacterial fluids, or irrigation fluids. Various wound therapy systems and components are commercially available through KCI USA, Inc. of San Antonio, Tex., U.S.A.

Conduit 44 can comprise a single lumen conduit (e.g., switched between a vacuum source and/or a fluid source) or can comprise multiple single-lumen conduits or a multi-lumen conduit such that, for example, fluid can be delivered and/or negative pressure can be applied to wound insert 20 individually or simultaneously. Or conduit 44 can comprise multiple lumens, for example, as in a single conduit with a central limit for application of negative pressure and/or fluid delivery and one or more peripheral lumens disposed adjacent or around the central lumen such that the peripheral lumens can be coupled to a pressure sensor to sense and/or detect a pressure or negative pressure between drape 40 and wound surface 52. In the embodiment shown, system 100 further comprises a wound dressing connection pad 42 configured to be coupled (and is shown coupled) to conduit 44. One example of a suitable connection pad 42 is the “V.A.C. T.R.A.C.® Pad,” commercially available from KCI USA, Inc. of San Antonio, Tex., U.S.A. One example of a suitable drape 40 includes the “V.A.C.® Drape” commercially available from KCI USA, Inc. of San Antonio, Tex., U.S.A.

In the embodiment shown in FIG. 1, apparatus 60 may be configured to deliver instillation fluid to wound 50, to remove fluid from wound 50, or to apply negative pressure to wound 50 through drape 40 and wound insert 20, or any combination of the three. Together, drape 40 and wound insert 20 may be referred to as a wound dressing 38. A fluid source in apparatus 60 may be activated to deliver fluid, such as saline, to wound surface 52 through conduit 44 coupled to wound insert 20 through connection pad 42. Alternatively, a vacuum source in apparatus 60 may be actuated to provide negative pressure to wound 50 and wound surface 52 through drape 40 and wound insert 20.

Example of instillation fluids that may be delivered to wound 50 are hypochlorous acid (HOCl) and hypochlorite ion (ClO—, which is also commonly referred to, generally understood to be synonymous with, and may be referred to interchangeably in this disclosure as, OCl—), which are examples of effective antimicrobial agents for biocidal action. For example, HOCl is typically capable of killing a broad spectrum of microbes (e.g., fungus, bacteria, viruses, fungus, yeast, and the like); often in a relatively short period of time (e.g., is capable of killing greater than 99% of microbes within a period of less than 10 seconds). Such antimicrobial agents can be generated or formed by a combination of the present reactive agents and fluid (e.g., water and/or aqueous solution, such as, for example, saline solution) and may be more effective and/or more versatile than antibiotics and other commonly used antimicrobial agents used in wound treatment in the past. For example, antibiotics may be bacteria-specific such that testing may be required to determine a suitable antibiotic to use for a specific wound or infection; and/or such that antibiotics may have only limited effectiveness for individual wounds and/or infections (e.g., where testing is not performed and/or where a wound is infected with a plurality of different bacteria). Such testing may take as long as several days to determine an appropriate antibiotic, delaying treatment or selection of an effective antibiotic. Additionally, bacteria may develop resistance to antibiotics, such that antibiotics may have reduced effectiveness after an amount of time. Further, antibiotics are typically administered intravenously (systemically) such that antibiotics may kill beneficial bacteria (e.g., in a patient's digestive system) and/or may cause organ damage (e.g., to a patient's liver).

Further, apparatus 60 may be configured to remove spent instillation fluids, secretions (e.g., pus), and/or infected tissue from wound 50. Undesirable effluent may be removed by actuating the vacuum source in apparatus 60; effluent may flow into wound insert 20, through conduit 44, and into a waste chamber coupled to apparatus 60.

Wound insert 20 may be of any suitable shape, including a rectangular prism or a cylinder. In certain applications, wound insert 20 may comprise a regular or irregular polygon having a depth, depending on the shape of the wound into which it is intended to be inserted. One of skill in the art will appreciate that the dimensions of wound insert 20 in the accompanying figures have been exaggerated for clarity.

Wound insert 20 may comprise closed-celled foam or open-celled foam. In certain specific embodiments, wound insert 20 comprises open-celled reticulated foam. Open-celled reticulated foam has a netlike microstructure, with few if any closed cells. In certain embodiments, the porosity can range from 95%-98%, though less porous or more porous foams may be used. In certain embodiments, wound insert 20 may comprise various polymers, which may include a polyurethane, such as polyurethane-polyester or polyurethane-polyether; polyolefins, such as polypropylenes (PP) or polyethylenes (PE); silicone polymers; polyvinylchloride; polyamides; polyesters; acrylics; thermoplastic elastomers such as styrene-butene-styrene (SBS) or styrene-ethylene-butene-styrene (SEBS); polyether-amide block copolymers (PEBAX); elastomers such as styrene butadiene rubber (SBR); ethylene propylene rubber (EPR); ethylene propylene diene modified rubber (EPDM); natural rubber (NR); ethylene vinyl acetate (EVA); polyvinyl alcohol (PVOH); polyvinyl acetal; or polyvinyl butyral (PVB). Additionally, wound insert 20 may comprise a bioabsorbable polymer, examples of which include polylactic acid, polylactide (PLA), polyglycolic acid, polyglycolide (PGA), and polycaprolactone (PCL). Methods of manufacturing open-celled reticulated foam are well known. Open-celled reticulated foam is commercially available from a variety of sources, including Kinetic Concepts, Inc., San Antonio, Tex., <www.kcil.com>, 1-800-275-4524.

In certain embodiments, wound insert 20 is configured to act as a negative pressure manifold. That is, wound insert 20 is configured to distribute negative pressure from a negative pressure source across wound 50, wound surface 52, or both. While various embodiments of wound insert 20 comprise foam or foam members, other structures may be used to distribute negative pressure across wound 50, wound surface 52, or both.

In certain embodiments, wound insert 20 is detectable by a hand-held metal detector. In certain specific embodiments, wound insert 20 is coated with a metal, including for example, metallic particles. In other specific embodiments, metal or metallic particles are mixed with the polymer wound insert 20 during the manufacturing process such that the metallic particles are fully incorporated into the microstructure of wound insert 20. Suitable metallic particles may comprise silver, iron, cobalt, gadolinium, europium, yttrium, chromium, and nickel, as well as their alloys and oxides. One example of a suitable embodiment of a wound insert is the Granufoam Silver wound insert, commercially available from Kinetic Concepts, Inc., San Antonio, Tex., <www.kcil.com>, 1-800-275-4524.

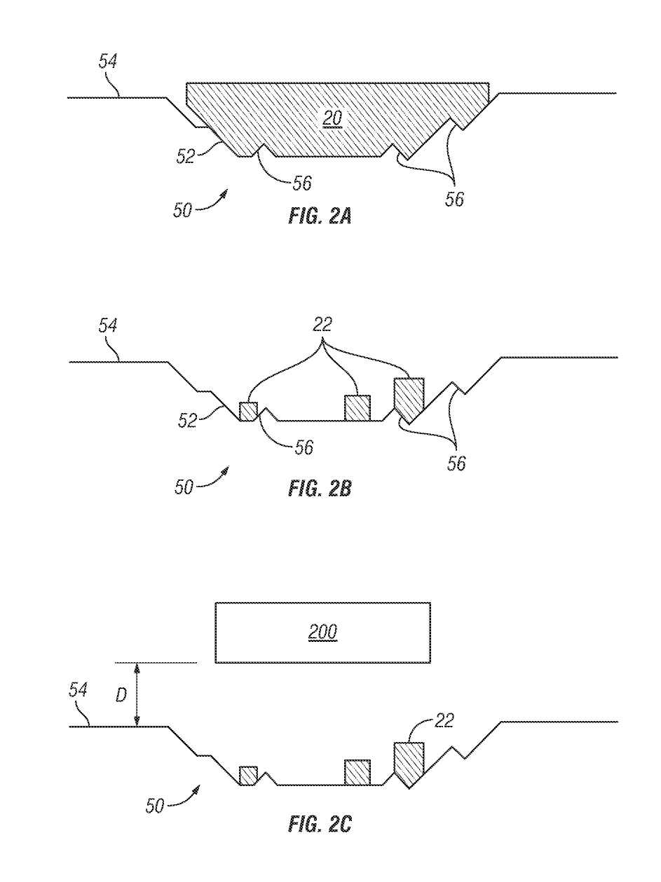

The delivery of instillation fluids and the application of negative pressure to wound 50 encourages the growth of granulation tissue 56 at wound boundary 52. Dressing 38 must be changed regularly to prevent wound insert 20 from becoming clogged with fibrin, exudate, effluent, or other wound materials, and to prevent infection at wound 50. As shown in FIG. 2A, when wound insert 20 is left in wound 50 for too long or when dressing 38 is not changed with sufficient regularity, granulation tissue 56 may grow into wound insert 20, becoming enmeshed with the insert.

As shown in FIG. 2B, when wound insert 20 is removed from wound 50, remnants 22 of wound insert 20 may be left in wound 50. In certain instances, these remnants 22 are separated from wound insert 20 by granulation tissue 56 that has become enmeshed with the wound insert 20. In other cases, such as where wound 50 is large, several wound inserts 20 may be used to fill wound 50. It is possible that a doctor, nurse, or other operator may neglect to remove all wound inserts 20 from wound 50. Wound 50 may become infected if wound inserts 20 or remnants 22 are allowed to remain within wound 50 as it heals. Wound inserts 20 and remnants 22 can be generally referred to as “wound interface materials,” and the term “wound interface materials” should be understood to encompass wound inserts 20, remnants 22, or both.

A metal detector 200 may be used to ensure that all wound interface materials 24 have been removed from wound 50. Metal detector 200 generates an electromagnetic field, and as the electromagnetic field passes over a magnetically conductive object (e.g., wound interface materials 24), the object creates an electromagnetic field of its own. Metal detector 200 is configured to detect the presence of the electromagnetic field generated by the magnetically conductive objects. For example, as shown in FIG. 2C, a hand-held battery-powered metal detector 200 may be used. Suitable hand-held metal detectors 200 include the Zircon MT6, the Stanley MetalSensor, and the Rapitest Cable and Stud detector. Other metal detectors may be used, including metal detectors that are configured to be coupled to a floor support, ceiling support, bed frame support, or other stationary support, as well as metal detectors that require an electrical current source. Very low frequency metal detectors, pulse induction metal detectors, and beat-frequency oscillator metal detectors may be used.

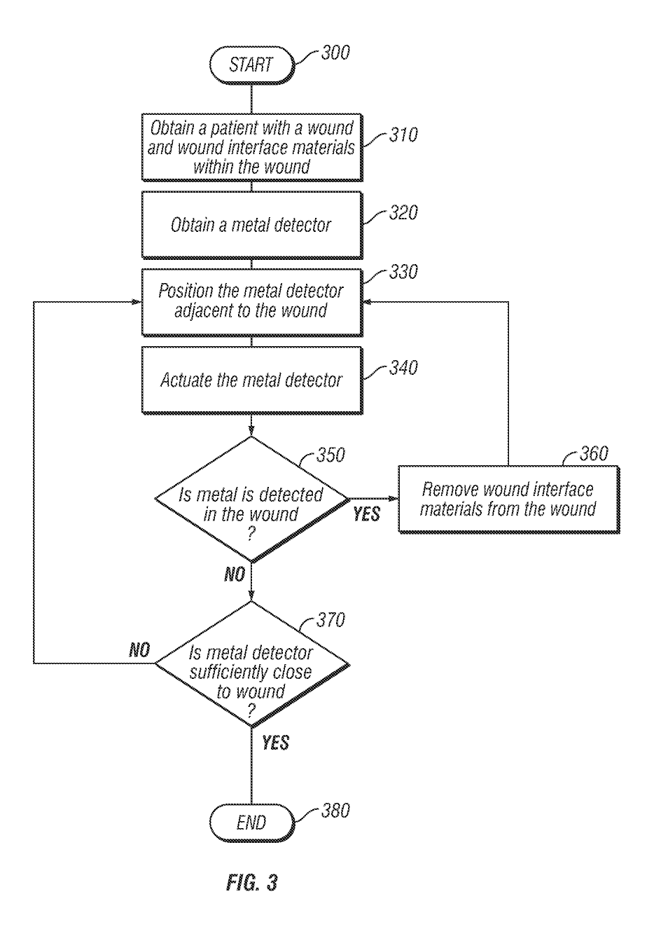

FIG. 3 illustrates one embodiment of a method for remotely detecting wound interface materials 24 in wound 50. A patient is obtained having wound 50 and wound interface materials 24 within wound 50. A metal detector 200 is also obtained.

Metal detector 200 may be positioned at a distance D (shown in FIG. 2C) over wound 50. Distance D may be less than about 1 in., 2 in., 3 in., 4 in., 5 in., 6 in., 7 in., 8 in., 9 in., 10 in., 11 in., 12 in., 13 in., 14 in., 15 in., 16 in., 17 in., 18 in., 19 in., 20 in., 21 in., 22 in., 23 in., or 24 in, or any suitable distance where metal detector 200 can detect the presence of metal. In specific embodiments, an operator (e.g. a doctor, nurse, technician, etc.) may hold metal detector 200 at distance D from wound 50. In other embodiments, metal detector 200 may be coupled to a positionable arm, which arm may be positioned in a desired orientation such that metal detector 200 is at a distance D from wound 50.

Once in the desired position, metal detector 200 may be actuated. If metal detector 200 detects the presence of metal within wound 50, metal detector 200 will provide an indication of the presence of metal. In certain embodiments, the indication may be in the form of an audible signal (e.g., a beep, a buzz, a tone, etc.), a visual indication (e.g., a light, a digital display of signal strength and/or metal type, etc.), tactile feedback (a vibration, etc.) or any combination of the three. In specific embodiments, the sensitivity of metal detector 200 may be increased such that metal detector 200 can better detect the presence of metal without decreasing distance D between metal detector 200 and wound 50.

In still other embodiments, metal detector 200 may be configured to display, transmit, or otherwise indicate the type of metal detected. In such embodiments, wound insert 200 may be distinguished from other metal objects that may be present in or near the patient's body, such as staples, plates, implants, etc, provided that the metal present in wound insert 200 differs from the other metal.

In alternate embodiments, metal detector 200 may be configured to display whether wound interface materials have been detected. For example, wound insert 20 may comprise metallic particles of a certain alloy having a specific signature. Metal detector 200 may be configured to detect the specific signature of the alloy and indicate that wound interface materials are present in wound 50. For example, metal detector 200 may comprise a screen or may be coupled to a screen configured to display a message to a user such as “WOUND INSERT DETECTED” when wound interface materials are detected in the wound. In this way, an operator may be able to distinguish between wound interface materials and materials that may provide a false positive reading (e.g., staples, plates, implants, etc.) in other metal detectors.

If metal detector 200 indicates that no metal is present within the wound 50, distance D between metal detector 200 and wound 50 may be decreased, the sensitivity of metal detector 200 may be increased, or both. Metal detector 200 may be positioned over different portions of wound 50 closer to the wound, then actuated again.

If metal detector 200 indicates that metal is present within wound 50, an operator may inspect wound 50. If wound insert 20 is present within wound 50, it may be removed by the operator. If remnants 22 are present within wound 50, they may be removed by the operator; in some instances, such as where granulation tissue 56 has substantially grown into remnants 22, remnants 22 may be removed surgically.

The steps of positioning metal detector 200, actuating metal detector 200, inspecting wound 50, and removing wound inserts 20 or remnants 22 may be repeated as necessary until all wound interface materials 24 have been removed from wound 50. In some instances, metal detector 200 will fail to detect wound interface materials 24 present in wound 50. In such instances it may be necessary to reposition metal detector 200 to be closer to wound 50, to increase the sensitivity of metal detector 200, or both.

The various illustrative embodiments of devices, systems, and methods described herein are not intended to be limited to the particular forms disclosed. Rather, they include all modifications and alternatives falling within the scope of the claims.

The claims are not intended to include, and should not be interpreted to include, means-plus- or step-plus-function limitations, unless such a limitation is explicitly recited in a given claim using the phrase(s) “means for” or “step for,” respectively.

1. A method of detecting the presence of a wound interface material in a patient, the method comprising:
positioning a metal detector proximate to a wound surface of a patient; and
monitoring the metal detector to determine whether the metal detector indicates the presence of a negative pressure manifold in the patient.

2. The method of claim 1 further comprising:
removing a wound insert from a wound proximate to the wound surface prior to positioning the metal detector proximate to the wound surface of the patient.

3. The method of claim 2 wherein the wound insert comprises metallic particles.

4. The method of claim 3 wherein the metal detector is configured to detect and identify a type of metal comprised in the metallic particles.

5. The method of claim 3 wherein the type of metal comprised in the metallic particles is silver.

6. A method of detecting wound interface material in a wound comprising:
obtaining a metal detector at a first sensitivity setting;
positioning the metal detector at a first distance adjacent to a wound of a patient;
actuating the metal detector; and
observing whether metal is detected in the wound.

7. The method of claim 6 further comprising:
removing a wound interface material from the wound if metal is detected in the wound.

8. The method of claim 6, where the metal detector is a hand-held metal detector.

9. The method of claim 6, where the wound further comprises remnants of wound interface material, the method further comprising
adjusting the metal detector to a second sensitivity setting, where the second sensitivity setting is more sensitive than the first sensitivity setting;
actuating the metal detector;
observing whether the metal detector detects metal in the wound; and
removing remnants of wound interface material from the wound.

10. The method of claim 6, where the wound further comprises remnants of wound interface material, the method further comprising:
positioning the metal detector at a second distance adjacent to the wound, the second distance being smaller than the first distance;
actuating the metal detector;
observing whether metal is detected in the wound; and
removing remnants of wound interface material from the wound.

11. The method of claim 10, where removing further comprises surgically removing remnants of wound interface material from the wound.

12. The method of claim 6, further comprising wetting the wound insert.

13. The method of claim 6, where the metal detector has a volume less than 40 in³.

14. A method of detecting wound interface material in a wound comprising:
obtaining a patient with a wound and a dressing coupled to the wound, where the dressing comprises:
a drape coupled to the skin adjacent to the wound; and
a wound interface material comprising metallic particles between the drape and the wound;
where the dressing is further configured to be coupled to a negative pressure source;
removing the drape from the patient;
obtaining a metal detector at a first sensitivity setting;
positioning the metal at a first distance adjacent to the wound;
actuating the metal detector;
observing whether metal is detected in the wound; and
removing wound interface material from the wound.

15. The method of claim 14, where the metal detector is a hand-held metal detector.

16. The method of claim 14, where the wound further comprises remnants of wound interface material, the method further comprising
adjusting the metal detector to a second sensitivity setting, where the second sensitivity setting is more sensitive than the first sensitivity setting;
actuating the metal detector;
observing whether the metal detector detects metal in the wound; and
removing remnants of wound interface material from the wound.

17. The method of claim 14, where the wound further comprises remnants of wound interface material, the method further comprising:
positioning the metal detector at a second distance adjacent to the wound, the second distance being less than the first distance;
actuating the metal detector;
observing whether the metal detector detects metal in the wound; and
removing remnants of wound interface material from the wound.

18. The method of claim 17, where removing further comprises surgically removing remnants of the wound insert from the wound.

19. The method of claim 14, where the metal detector has a volume less than 40 in³.

20. A medical device comprising:
a metal detector configured to detect metal in a wound interface material proximal to a wound of a patient.

21. The device of claim 20, further comprising an indicator configured to provide an indication when metal is detected in a wound interface material proximal to a wound in a patient.

22. The device of claim 21, where the indicator emits an audible signal.

23. The device of claim 21, where the indicator emits a visual signal.

24. The device of claim 21, where the indicator provides tactile feedback.

25. The device of claim 21, further comprising a screen configured to display a message to a user that wound interface material has been detected.

26. A kit comprising:
a wound insert comprising a metal; and
a metal detector configured to detect the metal comprised in the wound insert.

27. The kit of claim 26, where the metal detector further comprises an indicator configured to provide an indication when metal is detected in the wound insert.

28. The kit of claim 27, where the indicator emits an audible signal.

29. The kit of claim 27, where the indicator emits a visual signal.

30. The kit of claim 27, where the indicator provides tactile feedback.

31. The kit of claim 27, further comprising a screen configured to display a message to a user that wound interface material has been detected.
